(19) United States
(12) Patent Application Publication
(10) Pub. No.: US 2011/0184470 A1
(43) Pub. Date: Jul. 28, 2011

(54) BONE SCREW ASSEMBLY

(75) Inventors: Josef Gorek, Ross, CA (US); Scott Jones, McMurray, PA (US)

(73) Assignee: K2M, INC., Leesburg, VA (US)

(21) Appl. No.: 13/057,843

(22) PCT Filed: Aug. 6, 2009
(86) PCT No.: PCT/US2009/052954

§ 371 Date: Mar. 15, 2011

Related U.S. Application Data
(60) Provisional application No. 61/188,173, filed on Aug. 7, 2008.

(51) Int. Cl. A61B 17/88; A61B 17/86

(52) U.S. Cl. 606/279; 606/305

(57) ABSTRACT

A bone screw assembly includes a first bone screw, second bone screw, and a coupler. The first bone screw defines a first axis and the second bone screw defines a second axis. The coupler is operably associated with the first bone screw and the second bone screw. The coupler is adapted to mount the first bone screw and the second bone screw to adjacent bone structures. The first axis of the first bone screw and the second axis of the second bone screw define an angle therebetween. The first bone screw and the second bone screw are securable to each other.

### CROSS REFERENCE TO RELATED APPLICATIONS

This application claims priority to, and the benefit of, U.S. Provisional Patent Application Ser. No. 61/188,173 filed Aug. 7, 2008, the entire contents of which are incorporated by reference herein.

### BACKGROUND

1. Technical Field

The present disclosure relates generally to orthopedic surgery with particular regard to spinal surgery. Specifically, the present disclosure relates to an apparatus and methods for fixating adjacent vertebral bodies relative to each other using bone screws.

2. Description of Related Art

There has been considerable interest and development in the field of spinal surgery to achieve spinal fusion, specifically in anterior column surgery. In the lumbar spine, for instance, there is now great interest in minimizing the morbidity associated with fusion procedures, i.e., the muscle stripping, denervation and/or devascularization that often accompany the more commonly utilized posterior approach fusion procedures. Posterior pedicle screw fixation can achieve this stability but also includes significant risk of injury to the spinal neural elements and damage to other spinal structures such as muscle, ligaments, facet joints etc. On the other hand, by accessing the spine via an anterior approach, which can be accomplished in a relatively atraumatic manner, the morbidity associated with the surgical approach can indeed be minimized, facilitating postoperative recovery.

Rigid stabilization of the vertebral segments being incorporated into the fusion is fundamental to reliably achieving a successful arthrodesis. As noted above, it is preferable to accomplish this stabilization in the least traumatic manner possible. In order to obviate the need for posterior spinal fixation anterior spinal fixation devices have often been utilized instead. These devices can effectively resist compressive forces, bending forces and translational forces (shear), but are less effective in resisting rotational and distraction (extension) forces.

Another concern related to these anterior adjunctive fixation devices is that the stability afforded by them is not always sufficient to eliminate the need for additional posterior fixation. Factors that limit the strength of the fixation include fixation at the bone screw interface. For instance, when the interrelationship of the plate to the screws is not in a “locked” relationship, the strength of the bony fixation is compromised. This is also true if the screws are of insufficient diameter and/or length or if the bone screws are parallel to one another rather than divergent. A limited number of screws also can adversely affect fixation.

Various companies provide low profile “stand alone” anterior fixation constructs. One such device is the STALIF™ device. The STALIF™ device combines a polyetheretherketone (“PEEK”) cage and three screws. The entire anterior fixation is contained within the disc space itself.

Another low profile intradiscal fixation option available is the SynFix-LR provided by Synthes Spine. This option combines a PEEK cage with a metal plate and four screws. The metal plate is configured such as to allow for two screws to be placed into the cephalad vertebral body and two screws into the caudad vertebral body.

### SUMMARY

The present disclosure is directed to a bone screw assembly including a first bone screw and a second bone screw. The first bone screw includes a shall having threads thereon and a head disposed at one end thereof. The head may have an opening for receiving the second bone screw. The opening in the head may have threads formed therein for engaging a head of the second screw. Alternatively, threads may be provided on the head of one screw with a lip formed in the bore of the other screw configured to receive the first screw, with the lip of the bore being formed from commercially pure titanium, while the threads on the head of the second screw may be formed of a harder titanium alloy. The first axis of the first bone screw and the second axis of the second bone screw may be disposed in an angular relationship between about 0° and about 180°. The first bone screw and the second bone screw may also be disposed in vertical registration. One of the bone screws may have a head with an opening for receiving the other bone screw. In this configuration, the first bone screw and the second bone screw may be threaded such that they are threadably engageable with each other. The head of one of the bone screws may include threading

It is contemplated that a coupler may be used in cooperation with the first and second bone screws. The coupler includes a pair of openings for receiving the first and second bone screws. At least one of the openings is configured for engaging the head of a bone screw such that the bone screw resists backing out of the opening. The coupler openings define an angle that is substantially 90°, although angles between about 0° and about 180° are contemplated. In particular, it is contemplated that the defined angle may be between about 30° and about 150°. Further still, it is also contemplated that the defined angle may be between about 60° and about 120°.

### BRIEF DESCRIPTION OF THE DRAWINGS

The above and other aspects and features of the present disclosure will become more apparent in light of the following detailed description when taken in conjunction with the accompanying drawings in which:

FIG. 1A is a perspective view of one embodiment of a bone screw assembly in accordance with the present disclosure;

FIG. 1B is an end view of the bone screw assembly of FIG. 1A;

FIG. 1C is a side cross-sectional view of the bone screw assembly of FIG. 1B taken along section line 1C-1C;

FIG. 2 is a perspective view of a first bone screw of the bone screw assembly of FIG. 1A;

FIG. 2A is a perspective view of an alternate embodiment of the first bone screw of FIG. 2;

FIG. 3 is a perspective view of a second bone screw of the bone screw assembly of FIG. 1A;

FIG. 4A is a perspective view of an alternate embodiment of a bone screw assembly according to the present disclosure;

FIG. 4B is an end view of the bone screw assembly of FIG. 4A;

FIG. 4C is a side cross-sectional view of the bone screw assembly of FIG. 4B taken along section line 4C-4C;

FIG. 5 is a perspective view of a first bone screw of the bone screw assembly of FIG. 4A;

FIG. 5A is a perspective view of an alternate embodiment of the first bone screw of FIG. 5;

FIG. 6 is a perspective view of a second bone screw of the bone screw assembly of FIG. 4A;

FIG. 7A is an end view of the bone screw assembly of FIG. 1A installed into adjacent vertebrae;

FIG. 7B is a partial side cross-sectional view of the bone screw assembly and vertebrae of FIG. 7A illustrating an intervertebral cage disposed between the adjacent vertebrae;

FIG. 8 is a perspective view of a coupler according to a further embodiment of the present disclosure;

FIG. 9 is a perspective anterior view of the coupler of FIG. 8 attached to adjacent vertebrae;

FIG. 10 is a perspective view of a transfacet fixation assembly with the bone screw assembly of FIG. 4A;

FIG. 11 is a side elevational view the transfacet fixation assembly of FIG. 10;

FIG. 11A is a side cross sectional view of a further embodiment of a bone anchor assembly;

FIG. 11B is a perspective view of the bone anchor assembly of FIG. 11A;

FIG. 12 is a perspective view of a further embodiment of the transfacet fixation assembly with the first bone screw of FIG. 5, a polyaxial bone screw, and a cross connector;

FIG. 12A is a perspective view of the polyaxial bone screw of FIG. 12;

FIG. 12B is a perspective view of the transfacet fixation assembly of FIG. 12 implanted in a spine; and

FIG. 13 is a perspective view of another embodiment of the transfacet fixation assembly using the first bone screw of FIG. 5, the polyaxial bone screws of FIG. 12A, and a cross connector.

### DETAILED DESCRIPTION OF THE EMBODIMENTS

Particular embodiments of the present disclosure will be described herein with reference to the accompanying drawings. As shown in the drawings and as described throughout the following description, and as is traditional when referring to relative positioning on an object, the terms “proximal” and “trailing” may be employed interchangeably, and should be understood as referring to the portion of a structure that is closer to a clinician during proper use. The terms “distal” and “leading” may also be employed interchangeably, and should be understood as referring to the portion of a structure that is farther from the clinician during proper use. In addition, the term “cephalad” is used in this application to indicate a direction toward a patient's head, whereas the term “caudad” indicates a direction toward the patient's feet. Further still, the term “medial” indicates a direction toward the middle of the body of the patient, whilst the term “lateral” indicates a direction toward a side of the body of the patient (i.e., away from the middle of the body of the patient). The term “posterior” indicates a direction toward the patient's back, and the term “anterior” indicates a direction toward the patient's front. In the following description, well-known functions or constructions are not described in detail to avoid obscuring the present disclosure in unnecessary detail.

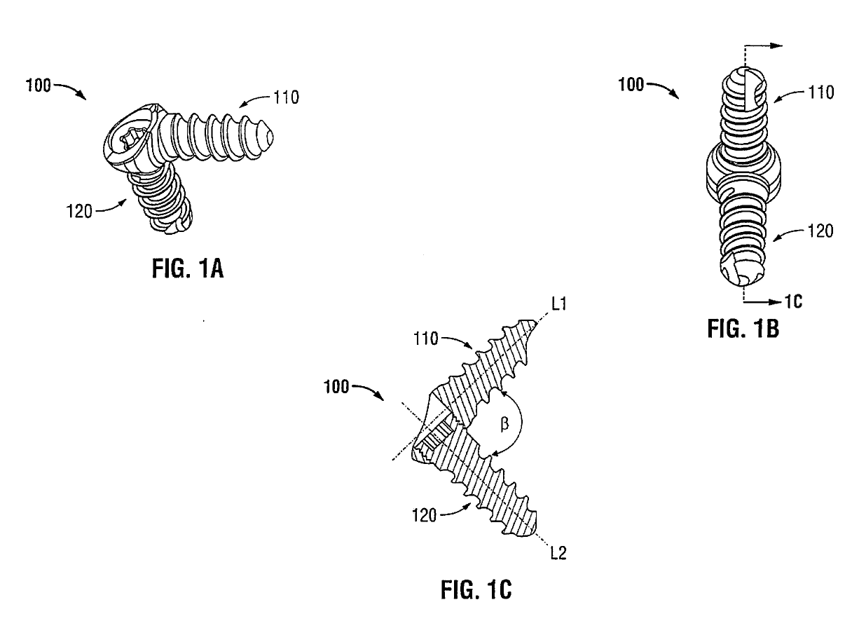

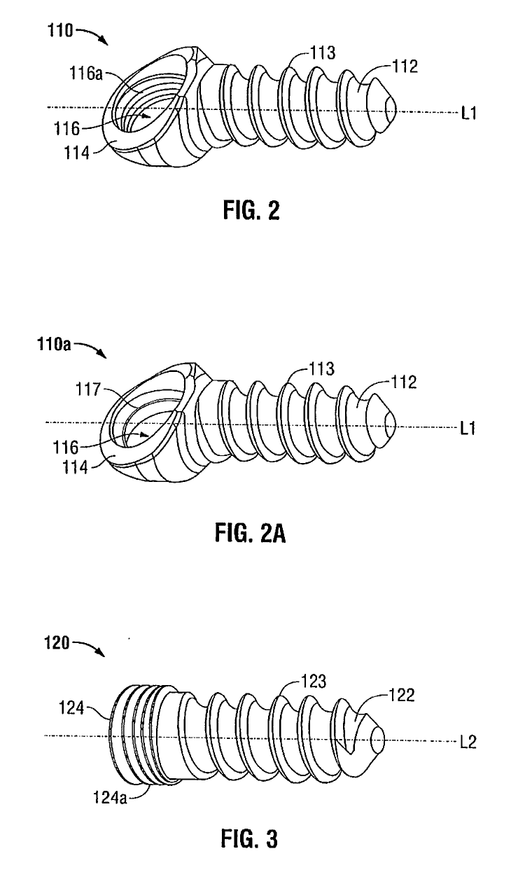

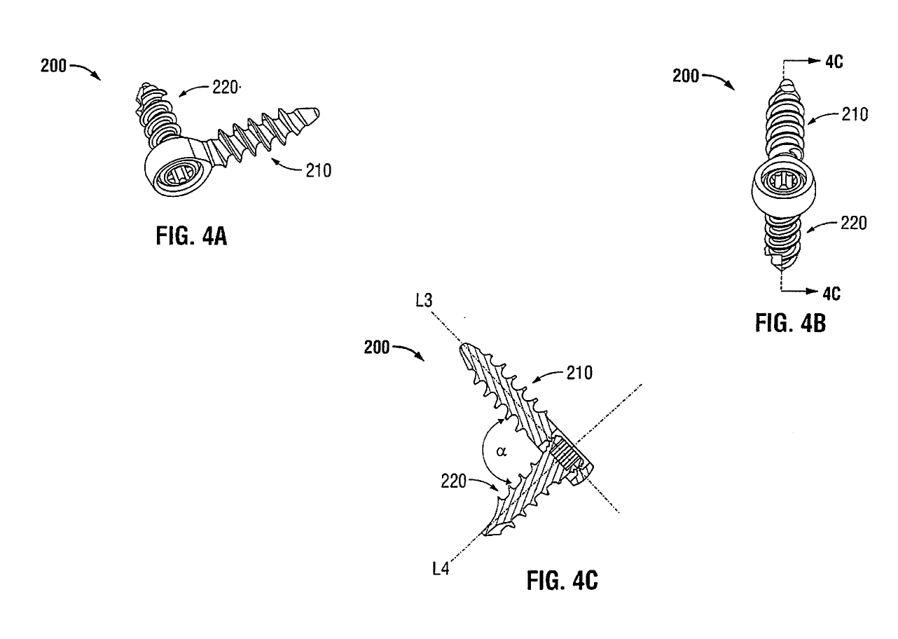

Referring initially to FIGS. 1-3, an embodiment of the presently disclosed bone screw assembly is shown and generally identified as 100. The bone screw assembly 100 includes a first bone screw 110 (FIGS. 2 and 2A) and a second bone screw 120 (FIG. 3). The first bone screw 110 includes a shall 112 and a head 114 attached thereto. The shaft 112 includes helical threads 113 peripherally disposed around the outer surface thereof. The threads 113 may adapted for threadably mating with cortical bone or with cancellous bone. The shall 112 of the first bone screw 110 defines a first longitudinal axis L1. A bore 116 extends through the head 114 of the first bone screw 110. The bore 116 is configured and dimensioned to accommodate at least a portion of the second bone screw 120 such that the first and second bone screws 110, 120 are coupled with each other as will be explained in further detail hereinbelow. The bore 116 of first bone screw 110 may include bore threads 116a (FIG. 2). Alternatively, first bone screw 110a may include a lip 117 (FIG. 2A). The bone screw 110 may be formed from any suitable biocompatible material such as titanium, titanium alloys, PEEK, or stainless steel. It is contemplated that all or a portion of the bone screw 110 may be formed from a resorbable material, as is known in the art. Further still, the bone screw 110 may be formed of several materials including metallic and polymeric materials. It is contemplated that the head 114 of the first bone screw 110 which includes bore 116 may be radially offset from the shaft 112.

The second bone screw 120 includes a shaft 122 and a head 124 attached thereto. The shaft 122 includes threads 123 peripherally disposed around the outer surface thereof. The threads 123, like threads 113 may adapted for threadably mating with cortical bone or with cancellous bone. It is contemplated that the shafts of the presently disclosed bone screws may be expandable or curved. The second bone screw 120 defines a second longitudinal axis L2. The first and the second longitudinal axes L1, L2 may be disposed at an angle β relative to each other. The angle β may be between about 0° to about 180°. In one embodiment, the angle β is about 45°. In another embodiment, the angle β may be between about 30° and about 150°, while in a further embodiment, the angle β may be between about 60° and about 120°. The head 124 of the second bone screw 120 includes head threads 124a for engaging the bore threads 116a of the head 114 of the first bone screw 110 and attaching the first bone screw 110 to the second bone screw 120 as will be discussed in detail hereinbelow. The pitch of the threads 123 on the shaft 122 of the second bone screw 120 when compared to the pitch of the head thread 124a on the head 124 of the second bone screw 120 can vary such as to allow for compression across the disk space. The head 124 may include a driver interface defined therein or projecting from an outer surface thereof for engaging with a driving tool, e.g. a screwdriver. The driver interface may be any suitable shape including circular, semi-circular, hexagonal, polygonal, etc.

The head 114 of the first bone screw 110 is configured for receiving the second bone screw 120 such that the first and second bone screws 110, 120 are affixed to each other. That is, the head 114 couples the first bone screw 110 with the second bone screw 120. The lip 117 of the head 114 of the first bone screw 110a is formed from commercially pure titanium. The threads 124a on the exterior of the head 124 of the second bone screw 120 are formed from a titanium alloy such as Ti-6Al-4V, which is harder than the commercially pure titanium of the lip 117. As such, since the commercially pure titanium of the lip 117 is softer than the Ti-6Al-4V alloy of the threads 124a of the screw head 124, the threads 124a engage the lip 117 as the head 124 of the second screw 120 is inserted through the bore 116, thereby inhibiting the second screw 120 from separating from the first screw 110a. It is further contemplated that alternate structures may be used to affix the first and second bone screws 110, 120. These alternate structures include clips, clamps, snaps, adhesives, etc. Alternatively, the threads 116a, 124a may be complementary for forming a secure attachment for the first and second bone screws 110, 120. Each head 114, 124 may be symmetrically or asymmetrically disposed relative to one or more of the shafts 112, 122 of the respective first and second bone screws 110, 120.

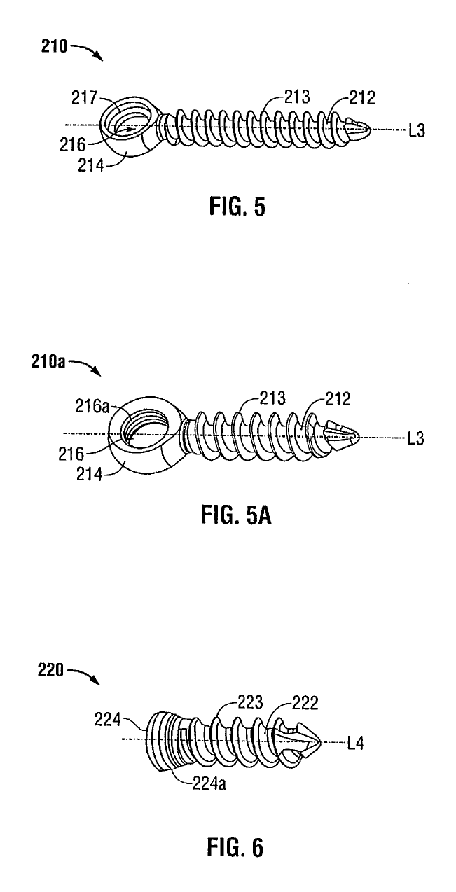

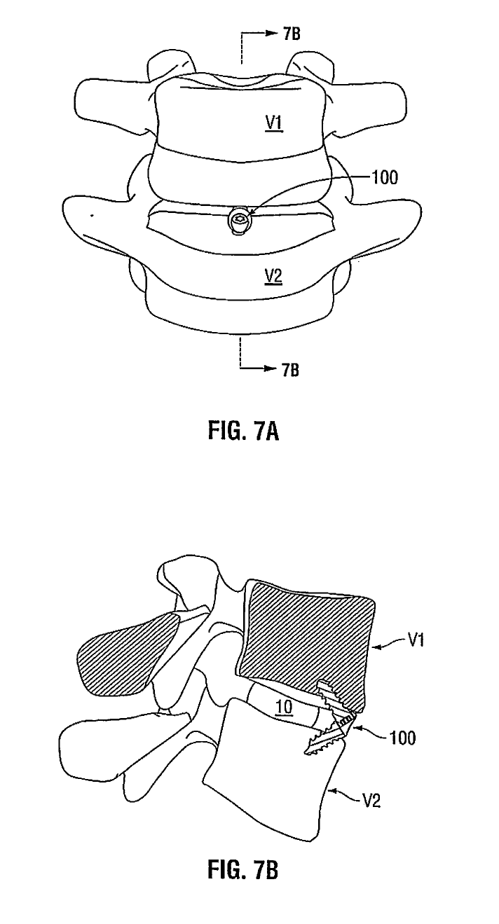

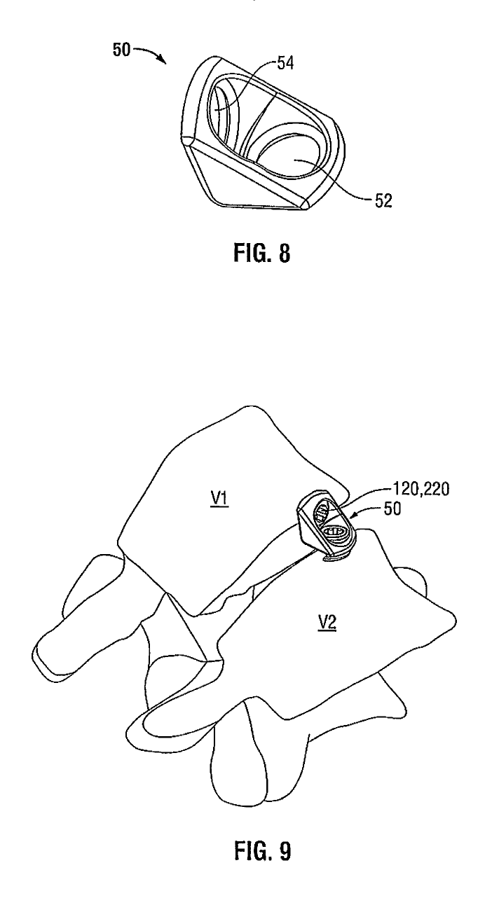

An alternate embodiment of the bone screw assembly is illustrated in FIGS. 4-6 and generally identified as bone screw assembly 200. Bone screw assembly 200 includes bone screws 210, 220. Bone screw 210 includes a shaft 212 with helical threads 213 formed on an outer surface thereof. A head 214 is attached to one end of the shaft 212. A bore 216 is extends through the head 214 of bone screw 210 and includes a lip 217 (FIG. 5) formed on an inner surface of the bore 216. Alternatively, the bore 216 of bone screw 210a may include threads 216a (FIG. 5A) formed on an inner surface thereof. The shaft 212 of the bone screw 210 defines a longitudinal axis L3, while the shaft 222 of bone screw 220 defines a longitudinal axis L4. The longitudinal axes L3, L4 define an angle α therebetween. As shown, the angle α is about 90°, although other angular relationships are within the scope of the present disclosure. Bone screw 220 includes a shaft 222 with helical threads 223 formed on an outer surface thereof. A head 224 is attached to one end of the shaft 222. The head 224 includes threads 224a formed on an outer surface thereof. The pitch of the threads 223 on the shaft 222 of the second bone screw 220 when compared to the pitch of the head thread 224a on the head 224 of the second bone screw 220 can vary such as to allow for compression across the disk space. The head 224 may include a driver interface defined therein or projecting from an outer surface thereof for engaging with a driving tool, e.g. a screwdriver. The driver interface may be any suitable shape including circular, semi-circular, hexagonal, polygonal, etc.

The lip 217 of the head 214 of the first bone screw 210 is formed from commercially pure titanium. The threads 224a on the exterior of the head 224 of the second bone screw 220 are formed from a titanium alloy such as Ti-6Al-4V, which is harder than the commercially pure titanium of the lip 217. As such, since the commercially pure titanium of the lip 217 is softer than the Ti-6Al-4V alloy of the threads 224a of the screw head 224, the threads 224a engage the lip 217 as the head 224 of the second screw 220 is inserted through the bore 216, thereby inhibiting the second screw 220 from separating from the first screw 210. It is further contemplated that alternate structures may be used to affix the first and second bone screws 210, 220. These alternate structures include clips, clamps, snaps, adhesives, etc. Alternatively, the threads 216a, 224a may be complementary for forming a secure attachment for the first and second bone screws 210a, 120. Each head 214, 224 may be symmetrically or asymmetrically disposed relative to one or more of the shafts 212, 222 of the respective first and second bone screws 210, 220. Advantageously, the lip-thread interlocking arrangement of the embodiment of FIGS. 4-6 permits the second screw to engage the bore a variety of angles, such that the axes L1 and L2 may be disposed at an angle relative to each other, providing greater flexibility to the surgeon during insertion of the screws into bone. An example of this locking arrangement is disclosed in U.S. Pat. No. 6,322,562 to Wolter, the entire contents of which are hereby incorporated by reference.

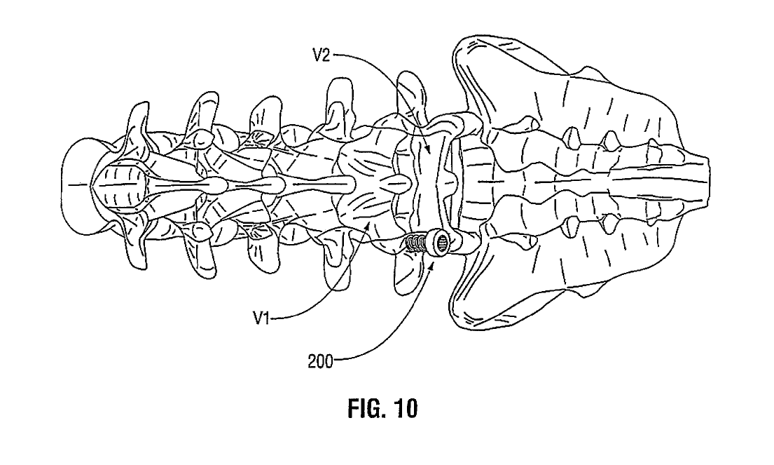

Referring additionally to FIGS. 7A and 7B, the bone screw assembly 100 is coupled to adjacent vertebral bodies V1, V2. Although only bone screw assembly 100 is illustrated and discussed with respect to vertebral bodies V1, V2, it is within the scope of the present disclosure that bone screw assembly 200 may be used with vertebral bodies V1, V2 in lieu of bone screw assembly 100. The first bone screw 110 is inserted into a previously drilled hole extending into one of the vertebral bodies V1, V2. Alternatively, the bone screw 110 may include self-starting threads such that little or no pre-drilling is required. The shaft 122 of the second bone screw 120 is directed through the bore 116 of the first bone screw 110 with the threads 123 of the second bone screw 120 engaging a previously drilled hole extending into one of the vertebral bodies V1, V2. Alternatively, the second bone screw 120 may include self-starting threads such that little or no pre-drilling is required. After insertion of the second bone screw 120, the heads 114, 124 of the first and second bone screws 110, 120 are then interlocked, as discussed hereinabove, such that the first and second bone screws 110, 120 are disposed at an angle β relative to each other. The bone screws 110, 120 are selected such that the respective shafts 112, 122 have sufficient length such that the bone screws 110, 120 are securely affixed to their respective vertebral bodies V1, V2 (i.e. sufficient purchase into bone tissue). Further still, since the bone screws 110, 120 are inserted at a preselected angle and the head 124 of bone screw 120 is securely affixed to the head 114 of bone screw 110, both bone screws 110, 120 resist separation from vertebral bodies V1, V2. As such, the first and second bone screws 110, 120 affix the first and second vertebral bodies V1, V2 in a relative relationship with each other. It is contemplated that an intervertebral implant 10 may be disposed between the vertebral bodies V1, V2 prior to attaching the bone screw assembly 100, 200 to the vertebral bodies V1, V2. It is contemplated that the intervertebral implant 10 may be retained in position by the bone screw assembly 100, 200 or that it may be threadably engaged with the bone screw assembly 100, 200. It is contemplated that the intervertebral implant 10 may include one or more openings for receiving the shafts of the screws of the bone screw assembly. The openings in the intervertebral implant may be complementary to the thread of the bone screws of the selected bone screw assembly. Alternatively, the interbody implant may be contoured to nest with the screw assembly.

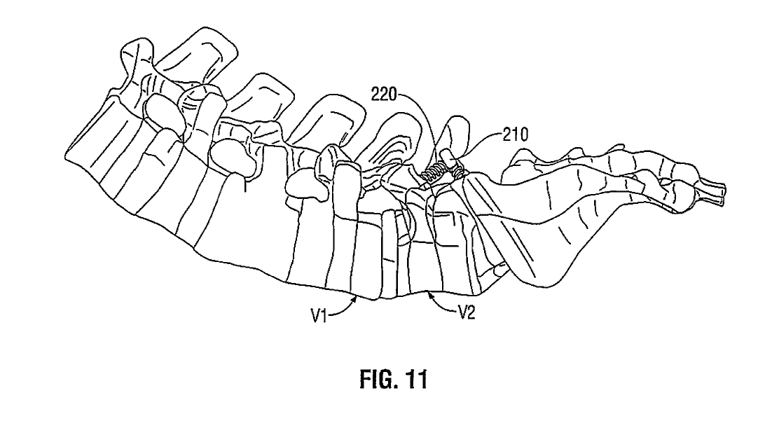

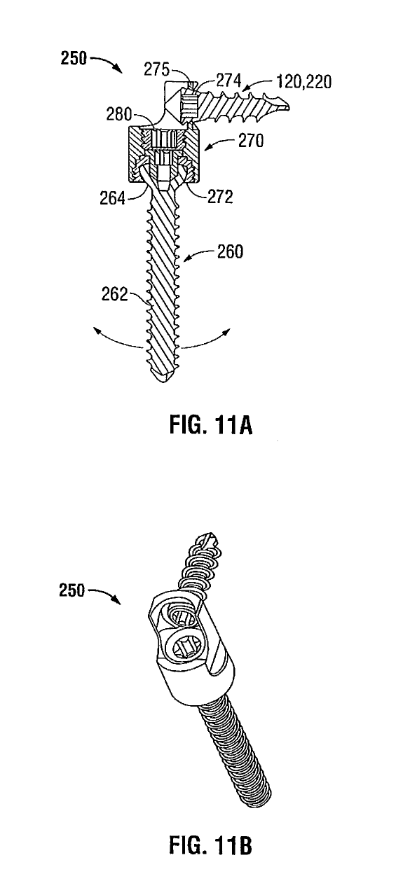

Referring now to FIGS. 8 and 9, in an alternate embodiment a coupler 50 (FIG. 8) includes first and second bores 52, 54 defined therethrough. Each bore 52, 54 is adapted to receive a bone screw 110, 120, 210, 220 for mounting to vertebral bodies V1, V2 and includes respective lips 52a, 54a. First and second bores 52, 54 are shown disposed in substantially orthogonal relationship. However, first and second bores 52, 54 may be disposed at any suitable angle relative to one another for receiving bone screws therethrough. Similar to the fastening arrangement discussed hereinabove with respect to bone screw assembly 100, 200, at least one of the lips 52a, 54a is formed from commercially pure titanium. When the second bone screw 120, 220 is inserted through the bore 52, 54 and engages the lip 52a, 54a that is formed from commercially pure titanium, the threads 114a, 124a that are formed from a harder titanium alloy such as Ti-6Al-4V engage the threads 114a, 124a and affix the bone screw 120, 220 to the bore 52, 54 of the coupler 50. In this arrangement, the bone screw 120, 200 is resistant to backing out of the bore 52, 54. Bone screw 110, 210 is inserted through the other bore 52, 54 and into the bone of the vertebral body. When the coupler 50 is assembled with bone screws 120, 220, the assemblage fixates the adjacent vertebral bodies V1, V2 with respect to each other. It is also envisioned that when bone screws 120, 220 are installed in the coupler 50, one bone screw 120, 220 may be partially threaded into the bore 52, 54 such that the head 124, 224 covers the head 124, 224 of the remaining bone screw 120, 220, thereby limiting the distance the remaining bone screw 120, 220 can travel in the event it starts to back out of the coupler 50. It is contemplated that the connector may be configured to nest with an interbody implant or with a bone plate mounted to the vertebral bodies above and below the intervertebral space. It is also contemplated that the presently disclosed coupler may have a hinge located between the bores or that the coupler is flexible such that the angular relationship between the bores is adjustable.

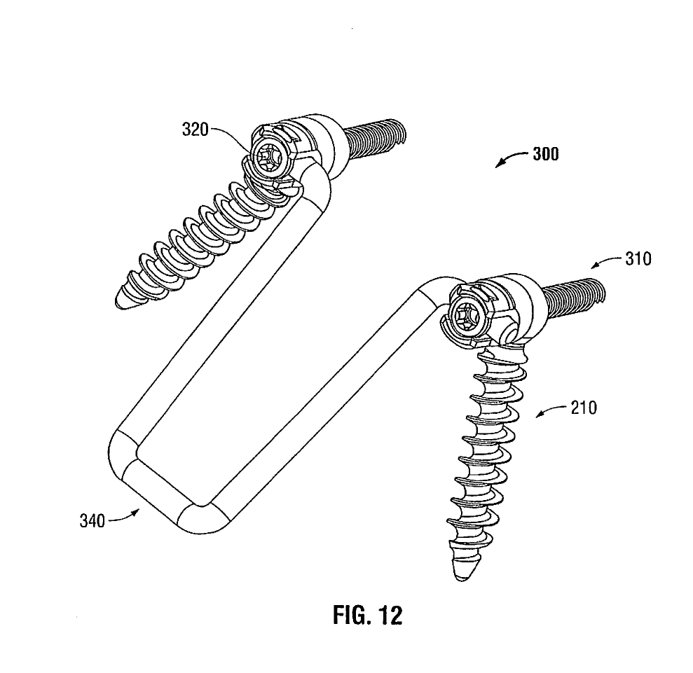

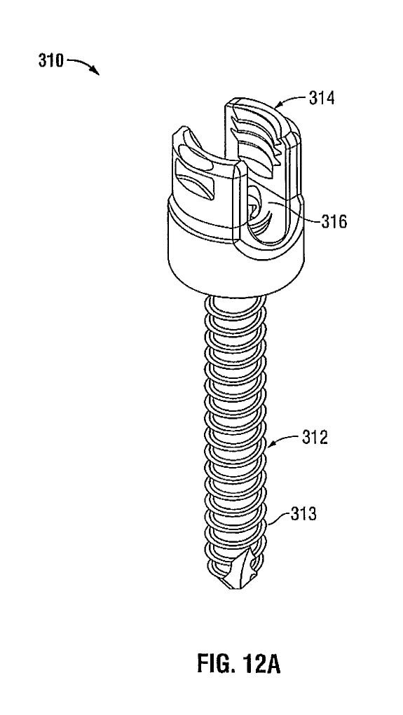

Either of the presently disclosed bone screw assemblies 100, 200 are capable of being used for transfacet fixation. Referring additionally to FIGS. 10 and 11, bone screw 210 is inserted into the pars articularis PA. Bone screw 220 is inserted through head 214 of bone screw 210 and inserted into the facet joint. Alternatively, bone screw 210 may be inserted into the facet joint and bone screw 220 is received through head 214 of bone screw 210 and secured to the pars articularis PA. It is contemplated that either bone screw 210 or 220 may be inserted towards the midline into the spinolaminar junction or spinous process or lamina. These sites would provide additional bony purchase on the vertebral body. It is envisioned that the bone screw 210 may be inserted through a pre-drilled hole or may have self-starting threads formed thereon as discussed hereinabove. Since the pars articularis PA is formed from cortical bone, it provides a much more secure purchase for the bone screw 210. Once bone screw 210 is securely anchored in the pars articularis PA, the other bone screw 220 is inserted through the head 214 of bone screw 210 and affixed through the facet joint as discussed hereinabove. The location of the installation of the bone screw assembly 200 into the pars articularis PA provides a secure anchor point. As such, this arrangement provides a more stable fixation arrangement for V1, V2 with respect to each other than provided by using a transfacet screw alone. Further still, this arrangement allows the practitioner to build other constructs.

Alternatively, with reference to FIG. 11A, a bone anchor 250 may be used in place of bone screw 210. Bone anchor 250 includes a polyaxial bone screw 260 and a coupling member 270. The bone screw 260 includes a head 264 and a shaft 262. The shaft 262 includes threads 263 peripherally disposed on an outer surface thereof. The head 264 has an arcuate portion that is proximal to the shaft 262. The bone screw 260 is rotatable relative to the coupling member 270 and is also repositionable such that a plurality of angular relationships may be defined between the bone screw 260 and the coupling member 270. The coupling member 270 includes a plurality of openings 272, 274 extending therethrough. Opening 272 is configured and dimensioned for receiving the head 264 of the bone screw 260 therethrough such that the head 264 is pivotably disposed in the opening 272. The coupling member 270 further includes threads 273 formed on an inner surface thereof for threadably engaging a set screw 280. As set screw 280 is threaded towards the head 264 of the bone screw 260, it frictionally engages the head 264 and secures the bone screw 260 in a set orientation relative to the coupling member 270. The other opening 274 is disposed orthogonally to opening 272. Opening 274 includes a lip 275 formed on an inner surface thereof. Bone screw 120 or bone screw 220 is insertable through the opening 274. The lip 275 is formed from commercially pure titanium. The threads 124a or 224a on the exterior of the head 124 or 224 of the bone screw 120 or 220 are formed from a titanium alloy such as Ti-6Al-4V, which is harder than the commercially pure titanium of the lip 275. As such, since the commercially pure titanium of the lip 275 is softer than the Ti-6Al-4V alloy of the threads 124a, 224a, the threads 124a, 224a engage the lip 275 as the head 124, 224 of the bone screw 120, 220 is inserted through the opening 274, thereby inhibiting the bone screw 120, 220 from separating from the bone anchor 250. As constructed, bone anchor 250 is insertable into a selected bone structure, such as a vertebral body, and the coupling member 270 is pivotable relative to the anchor location of the bone screw 260 allowing the coupling member 270 to be pivoted and/or rotated such that opening 274 is in a desired orientation to the target bone structure. Bone screw 120 or 220 is inserted through the opening 274 and affixed to the target bone structure. The head 124 or 224 is secured to the lip 275 of the opening 274 as discussed above. As such, the target bone structure is fixedly coupled to the selected bone structure. It is contemplated that the bone structures may include pars articularis, spinolaminar junction, spinous process, or lamina.

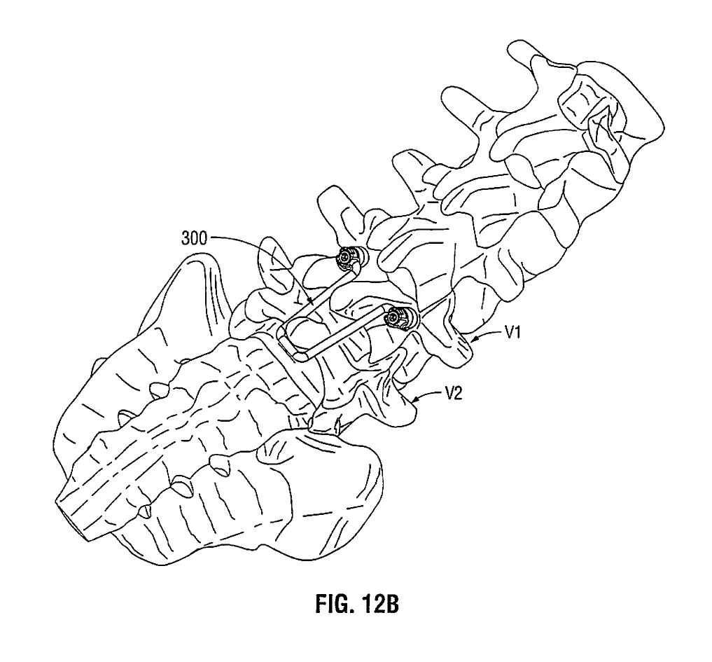

An example of one additional construct is illustrated in FIGS. 12 and 12A. Bone fixation assembly 300 includes bone screw 210, a bone screw 310 (FIG. 12A), and a cross connector 340 such as a cable, a tension band, or a rod. The cross connector 340 may be rigid or may have a predetermined amount of resiliency. Bone screw 310 includes a shaft 312 having threads 313 formed thereon. The bone screw 310 includes a head 314 having a generally U-shaped saddle 316. The U-shaped saddle 316 includes threads formed on an inner surface thereof. A setscrew 320 (FIG. 12) is threadably engageable with the threads of the saddle 316 for securing a portion of the cross connector 340 within the confines of the saddle 316. The threads 313 are formed from a titanium alloy such as Ti-6Al-4V. Thus, when the bone screw 310 is inserted through the bore 216 of the bone screw 210, the titanium alloy of the threads 313 engage the softer commercially pure titanium of the lip 217 of the bone screw 210, thus securing the bone screws 210 and 310 to each other. Since the bone screw 210 is inserted into the cortical bone of the pars articularis PA, the addition of the bone screw 310 provides a secure anchor point for building the construct. This construct is repeated on the opposing side of the vertebral body V1, V2. Once the two constructs are positioned, the practitioner attaches one end of the cross connector 340 to one construct and the other end of the cross connector 340 to the other construct. The portion of the cross connector 340 between the opposing ends preferably is positioned to engage the spinous process SP of the adjacent vertebral body. Setscrews 320 are installed into the heads 314 of the bone screws 310, thereby securing the cross connector 340 to the constructs. As assembled in FIG. 12B, the bone fixation device 300 provides secure fixation of vertebral bodies V1, V2. Advantageously, the combination of a transfacet-pars articularis fixation combined with a cross connector such as a rod, tension band, or cable extending around the spinous process below the facet operated on to a similar transfacet-pars articularis construct at the same level on the opposite side of the spine provides enhanced stability to the spine. In particular, the cross connector is disposed around the spinous process of vertebral body V1. The cross connector provides additional rigidity to the construct and resists flexion, thereby relieving stress on the construct.

In yet a further embodiment it is contemplated that a construct may be made in which a transfacet-pars articularis construct includes a head on the second screw which is engageable with a rod, such as a titanium rod having a diameter of from about 3.5 mm to about 6 mm. The rod may extend to the adjacent vertebral level above or below the transfacet-pars articularis construct, where the rod may engage a similar transfacet-pars articularis construct or a pedicle screw, such as a polyaxial pedicle screw. It is contemplated that such a construct may provide enhanced spinal fixation by fixing adjacent levels of the spine by isolating and fixing the facet joint and fixing the adjacent spine level relative to the facet joint.

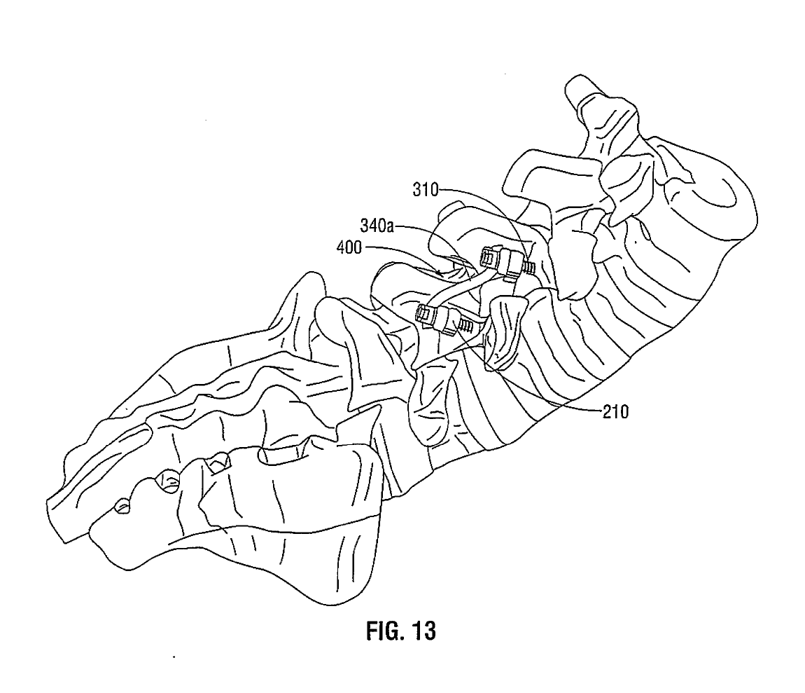

Referring now to FIG. 13, a further embodiment of the presently disclosed bone fixation assembly is illustrated. Bone fixation assembly 400 includes one or more bone screws 210, one or more bone screws 310, and a cross connector 340a. Similar to bone fixation assembly 300 (FIG. 12), bone screw 210 is inserted into the cortical bone of the pars articularis PA of vertebral body V1 and bone screw 310 is inserted through the head 214 into the facet joint. Since the threads 313 of bone screw 310 are formed of a titanium alloy Ti-6Al-4V, which is harder than the commercially pure titanium of the lip of 217 of the head 214 of bone screw 210, the threads 313 deform the lip 217 and secure bone screw 310 in the head 214 such that bone screw 310 resists backing out or separating from bone screw 210. Alternatively, head 214 may include threads that are complementary to the threads 313 such that when bone screw 310 is inserted through the head 214 of bone screw 210, the screws 210, 310 are threadably engaged such that they resist separation. In either embodiment, another bone screw 310 is inserted into an adjacent level facet joint or pars articularis. Cross connector 340a is shaped to match the orientation of the bone screws 310 and is inserted into the saddles 316 of the bone screws 310 and secured in position using setscrews 320.

It is contemplated that the coupling member or coupling portion of the bone screws may include multiple openings at varying angles to support multiple screw fixtures. Further, the presently disclosed bone screws are configured for attachment to various bone structures including pars articularis, spinolaminar junction, spinous process, or lamina.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. A bone screw assembly comprising:
a first bone screw defining a first axis;
a second bone screw defining a second axis; and
a coupler operably associated with the first bone screw and the second bone screw, the coupler being adapted to mount the first bone screw to the second bone screw, wherein the first axis of the first bone screw and the second axis of the second bone screw define an angle therebetween.

2. The bone screw assembly of claim 1, wherein the first bone screw is oriented orthogonally to the second bone screw.

3. The bone screw assembly of claim 1, wherein the coupler is disposed in mechanical cooperation with the first bone screw.

4. The bone screw assembly of claim 3, wherein the coupler is adapted to accommodate the second bone screw such that when the first bone screw is threaded through the coupler, the first and second bone screws are threadably locked to each other.

5. The bone screw assembly of claim 1, wherein the first axis and the second axis define an angle between about 0″ and about 180°.

6. The bone screw assembly of claim 3, wherein the coupler defines at least one coupler axis such that the at least one coupler axis and the first axis of the first bone screw define an angle between about 0° and about 180°.

7. The bone screw assembly of claim 6, wherein the coupler axis and the at least one second axis of the second bone screw are disposed in substantial axial alignment.

8. The bone screw assembly of claim 1, wherein at least one bore extends through the coupler that is adapted to accommodate the at least one second bone screw.

9. The bone screw assembly of claim 1, wherein a portion of the bone screw assembly is made of commercially pure titanium and a portion of the bone screw assembly is made of titanium alloy.

10. A method of mounting a bone screw assembly comprising:
providing a first bone screw, a second bone screw, and a coupler defining at least one bore extending therethrough, wherein the coupler is operably associated with at least one of the first and second bone screws;
mounting the first bone screw to a first vertebral body;
mounting the second bone screw to a second vertebral body; and
coupling the first bone screw and the at least one second bone screw via the coupler.

11. The method of claim 10, further including:
positioning a first axis of the first bone screw and a second axis of the second bone screw at an angle of between about 0° and about 180° with respect to one another.

12. The method of claim 10, further including:
mounting at least one of the first and second bone screws to an interbody cage implant.

13. The method of claim 10, wherein mounting the first bone screw includes mounting the first bone screw into a pars articularis of one vertebral body.

14. A method of spinal fixation comprising:
(a) inserting a first bone screw having a head including a bore into a facet joint;
(b) inserting a second bone screw through the bore and into bone adjacent to the facet joint;
(c) threading the second bone screw into the head of the first bone screw;
(d) attaching a coupling member to the second bone screw;
(e) repeating (a) though (d) on an opposing side of the same spinal level; and
(f) positioning a portion of the coupling member around a spinous process;
(g) securing the coupling member to the construct on the opposing side of the spine.

15. A method of treating bone comprising:
providing a first bone screw having a threaded shaft configured and dimensioned to engage bone and further having a coupler including a bore,
providing a second bone screw having a threaded shaft configured and dimensioned to engage bone, and further including a screw head having threads thereon;
mounting the first bone screw into bone;
mounting the second bone screw to bone by inserting the second screw threaded shaft through the bore and into bone; and
coupling the first bone screw to the second bone screw by engaging the threads of the second screw head with the coupler.

16. The method of claim 15 wherein the step of mounting the first bone screw comprises mounting the first bone screw to a facet joint.

17. The method of claim 16 wherein the step of mounting the second bone screw comprises mounting the second screw threaded shaft into the pars articularis adjacent the facet joint.

18. The method of claim 15 wherein the step of coupling includes engaging the second screw head threads with threads in the bore of the coupler.

19. The method of claim 15 wherein the step of coupling includes engaging the second screw head threads with a lip in the bore of the coupler, the coupler being formed of a softer material than the second screw.

20. A bone screw assembly comprising:
a first bone screw having a threaded shaft defining a first axis;
a second bone screw having a threaded shaft defining a second axis; and
a coupler operably associated with the first bone screw, the coupler being flexibly attached to the head of the first bone screw and having a bore configured and dimensioned to receive the second bone screw shaft therethrough.

21. The assembly of claim 20 wherein the bore includes threads and the second screw head includes threads configured and dimensioned to engage the bore threads.

22. The assembly of claim 20 wherein the bore includes a lip and the coupler is formed of a softer material than the second screw head, such that the threads of the second screw head are configured to deform the lip when rotatably engaged with the bore lip,

23. The assembly of claim 20 wherein the coupler is rotatably mounted to the first screw.

24. The assembly of claim 20 wherein the coupler is hingedly mounted to the first screw.
